(19) United States
(12) Patent Application Publication
(10) Pub. No.: US 2024/0125856 A1
(43) Pub. Date: Apr. 18, 2024

(54) METHOD FOR PREDICTING BATTERY PERFORMANCE BASED ON COMBINATION OF MATERIAL PARAMETERS OF BATTERY PULPING PROCESS

(71) Applicant: SHENZHEN INSTITUTES OF ADVANCED TECHNOLOGY, Shenzhen (CN)

(72) Inventors: Zhile YANG, Shenzhen (CN); Bangyu ZHOU, Shenzhen (CN); Chengke WU, Shenzhen (CN); Yuanjun GUO, Shenzhen (CN); Xiangfei LIU, Shenzhen (CN)

(73) Assignee: SHENZHEN INSTITUTES OF ADVANCED TECHNOLOGY, Shenzhen (CN)

(21) Appl. No.: 18/393,634

(22) Filed: Dec. 21, 2023

Related U.S. Application Data
(63) Continuation of application No. PCT/CN2022/137739, filed on Dec. 8, 2022.

(30) Foreign Application Priority Data
Oct. 8, 2022 (CN) 202211222968X

(51) Int. Cl. G01R 31/367; G06N 3/045; G06N 3/0985

(52) U.S. Cl. CPC G01R 31/367; G06N 3/045; G06N 3/0985

(57) ABSTRACT

The present disclosure discloses a method for predicting the battery performance based on a combination of material parameters of a battery pulping process, including obtaining a target prediction model; and obtaining a combination of material parameters to be predicted corresponding to the battery pulping process, and inputting the combination of the material parameters to be predicted into the target prediction model to obtain a target battery performance level corresponding to the combination of the material parameters to be predicted. The method of the present disclosure adopts a mathematical model method instead of manual testing, can quickly predict battery performance levels corresponding to different combinations of material parameters, and solves the problem that in the prior art, it is necessary to conduct separate tests for different input ratios of various materials to determine the impact of different material ratios on the battery performance.

### CROSS-REFERENCE TO RELATED APPLICATIONS

The application claims priority to Chinese patent application No. 202211222968X, filed on Oct. 8, 2022, the entire contents of which are incorporated herein by reference.

### TECHNICAL FIELD

The present disclosure relates to the field of model prediction, in particular to a method for predicting the battery performance based on a combination of material parameters of a battery pulping process.

### BACKGROUND

Ina process of improving the performance of lithium ion batteries, researchers have mostly focused on the research and modification of active materials, often neglecting the morphology of conductive agents and binders, and interactions of the conductive agents and the binders with the active materials. Although electrode materials can determine an upper limit of the battery performance, a battery pulping process will determine a lower limit of the battery performance, and therefore, the battery pulping process should be improved as much as possible so that the lower limit of the battery performance approaches the upper limit of the battery performance. The performance of batteries produced by different input ratios of various materials is different during the battery pulping process. In the prior art, it is usually necessary to conduct separate tests for different input ratios of various materials to determine the impact of different material ratios on the battery performance, which requires a lot of manpower and time costs.

Therefore, the prior art remains to be improved and developed.

### SUMMARY

### Technical Problem

The technical problem to be solved by the present disclosure is to provide a method for predicting the battery performance based on a combination of material parameters of a battery pulping process in view of the above defects in the prior art, aiming at solving the problem that in the prior art, it is necessary to conduct separate tests for different input ratios of various materials to determine the impact of different material ratios on the battery performance, which requires a lot of manpower and time costs.

### Technical Solution

The technical solution adopted by the present disclosure to solve the problem is as follows:
in a first aspect, an embodiment of the present disclosure provides a method for predicting the battery performance based on a combination of material parameters of a battery pulping process, wherein the method includes:
obtaining a target prediction model, wherein the target prediction model includes a plurality of sub-models, and the plurality of the sub-models correspond to different combinations of model parameters, respectively; and
obtaining a combination of material parameters to be predicted corresponding to the battery pulping process, and inputting the combination of the material parameters to be predicted into the target prediction model to obtain a target battery performance level corresponding to the combination of the material parameters to be predicted, wherein the target battery performance level is determined according to battery performance levels output by the sub-models based on the combination of the material parameters to be predicted, respectively.

In one embodiment, the target prediction model is trained in advance, wherein a training process includes:
obtaining a plurality of combinations of material parameters corresponding to the battery pulping process and actual battery performance levels respectively corresponding to the plurality of the combinations of the material parameters;
inputting one of the combinations of the material parameters into the target prediction model to obtain a first predicted battery performance level output by the target prediction model and a second predicted battery performance level output by each of the sub-models, wherein the first predicted battery performance level is determined based on the second predicted battery performance levels:
determining a loss value corresponding to each of the sub-models based on the first predicted battery performance level, the second predicted battery performance levels, and the actual battery performance level corresponding to the combination of the material parameters;
determining whether the loss value corresponding to each of the sub-models converges to a target value; and
if not, correcting a combination of initial model parameters of the sub-model according to the loss value of the sub-model not converging to the target value, and continuing to perform the step of inputting one of the combinations of the material parameters into the target prediction model until the loss value corresponding to each of the sub-models converges to the target value to obtain the trained target prediction model.

In one embodiment, determining the loss value corresponding to each of the sub-models based on the first predicted battery performance level, the second predicted battery performance levels, and the actual battery performance level corresponding to the combination of the material parameters includes:
determining a first loss value corresponding to each of the second predicted battery performance levels based on a deviation between the first predicted battery performance level and the actual battery performance level corresponding to the combination of the material parameters;
determining a second loss value corresponding to each of the sub-models according to a deviation between the second predicted battery performance level corresponding to each of the sub-models and the first predicted battery performance level; and
determining the loss value corresponding to each of the sub-models based on the first loss value and the second loss value corresponding to each of the sub-models.

In one embodiment, the method further includes determining a combination of target hyper-parameters corresponding to each of the sub-models before training the target prediction model, wherein a method for determining the combination of the target hyper-parameters corresponding to each of the sub-models includes:
obtaining a plurality of combinations of hyper-parameters, wherein each of the combinations of the hyper-parameters includes a plurality of hyper-parameters of different categories;
determining a plurality of hyper-parameter combination profiles according to the combinations of the hyper-parameters, wherein the plurality of the hyper-parameter combination profiles are in one-to-one correspondence to the plurality of the hyper-parameters, values of target hyper-parameters corresponding to each of the combinations of the hyper-parameters in each of the hyper-parameter combination profiles are the same, and each of the combinations of the hyper-parameters is regularly distributed based on the magnitude of the values of the target hyper-parameters, and the target hyper-parameters are the hyper-parameters corresponding to the combination of the hyper-parameters;
determining a combination of candidate hyper-parameters corresponding to each of the hyper-parameter combination profiles, wherein each of the combinations of the hyper-parameters adjacent to the combination of the candidate hyper-parameters in each of the hyper-parameter combination profiles has a model performance level that is less than a model performance level of the combination of the candidate hyper-parameters; and
determining the combination of the target hyper-parameters corresponding to each of the sub-models according to the combinations of the candidate hyper-parameters.

In one embodiment, determining the combination of the candidate hyper-parameters corresponding to each of the hyper-parameter combination profiles includes:
determining a target point based on one of the combinations of the hyper-parameters in each of the hyper-parameter combination profiles;
determining whether model performance levels respectively corresponding to points adjacent to the target point are greater than a model performance level corresponding to the target point;
if a model performance level corresponding to any one of the points adjacent to the target point is greater than the model performance level corresponding to the target point, determining a next target point according to a point having the highest model performance level in the points adjacent to the target point; and
continuing to perform the step of determining whether the model performance levels respectively corresponding to the points adjacent to the target point are greater than the model performance level corresponding to the target point until the model performance levels respectively corresponding to the points adjacent to the target point are less than the model performance level corresponding to the target point, and using the combination of the hyper-parameters corresponding to the target point as the combination of the candidate hyper-parameters corresponding to the hyper-parameter combination profile.

In one embodiment, determining the combination of the target hyper-parameters corresponding to each of the sub-models according to the combinations of the candidate hyper-parameters includes:
determining a target hyper-parameter combination profile from the plurality of the hyper-parameter combination profiles based on the model performance level corresponding to each of the combinations of the candidate hyper-parameters, wherein the combination of the candidate hyper-parameters corresponding to the target hyper-parameter combination profile has the highest model performance level;
traversing the target hyper-parameter combination profile to obtain first several combinations of the hyper-parameters having the highest model performance level, wherein the number of the first several combinations of the hyper-parameters is determined based on the number of the sub-models; and
determining, in one-to-one correspondence, the combination of the target hyper-parameters corresponding to each of the sub-models according to the first several combinations of the hyper-parameters.

In one embodiment, the target battery performance level is determined according to an average or a weighted average of the battery performance levels respectively corresponding to the sub-models.

In a second aspect, an embodiment of the present disclosure further provides an apparatus for predicting the battery performance based on a combination of material parameters of a battery pulping process, wherein the apparatus includes:
an obtaining module, configured to obtain a target prediction model, wherein the target prediction model includes a plurality of sub-models, and the plurality of the sub-models correspond to different combinations of model parameters, respectively; and
a prediction module, configured to obtain a combination of material parameters to be predicted corresponding to the battery pulping process, and input the combination of the material parameters to be predicted into the target prediction model to obtain a target battery performance level corresponding to the combination of the material parameters to be predicted, wherein the target battery performance level is determined according to battery performance levels output by the sub-models based on the combination of the material parameters to be predicted, respectively.

In a third aspect, an embodiment of the present disclosure further provides a terminal, wherein the terminal includes a memory and one or more processors; wherein the memory stores one or more programs including instructions for performing any one of the above methods for predicting the battery performance based on a combination of material parameters of a battery pulping process; and the processors are configured to execute the programs.

In a fourth aspect, an embodiment of the present disclosure further provides a computer-readable storage medium, having stored thereon a plurality of instructions adapted to be loaded and executed by a processor to perform the steps of any one of the above methods for predicting the battery performance based on a combination of material parameters of a battery pulping process.

### Beneficial Effects

Beneficial effects of the present disclosure: in the embodiments of the present disclosure, a mathematical model method is used instead of manual testing, battery performance levels corresponding to different combinations of material parameters can be quickly predicted, and the problem that in the prior art, it is necessary to conduct separate tests for different input ratios of various materials to determine the impact of different material ratios on the battery performance, which requires a lot of manpower and time costs is solved.

### BRIEF DESCRIPTION OF DRAWINGS

In order to illustrate the technical solutions in the embodiments of the present disclosure or the prior art more clearly, the drawings required for the description of the embodiments or the prior art will be briefly described below, and obviously, the drawings in the following description are only some embodiments of the present disclosure, and for those of ordinary skill in the art, other drawings can be obtained from these drawings without inventive steps.

FIG. 1 is a schematic flow diagram of a method for predicting the battery performance based on a combination of material parameters of a battery pulping process according to an embodiment of the present disclosure.

FIG. 2 is an internal block diagram of an apparatus for predicting the battery performance based on a combination of material parameters of a battery pulping process according to an embodiment of the present disclosure.

FIG. 3 is a functional block diagram of a terminal according to an embodiment of the present disclosure.

### DETAILED DESCRIPTION OF THE EMBODIMENTS

The present disclosure discloses a method for predicting the battery performance based on a combination of material parameters of a battery pulping process, and in order to make the objects, technical solutions and effects of the present disclosure more clear and definite, the present disclosure will be further described below in detail by way of embodiments with reference to the accompanying drawings. It should be understood that the specific embodiments described herein are merely illustrative of the present disclosure and are not intended to limit the present disclosure.

It will be understood by those skilled in the art that the singular forms “a”, “an”, “said”, and “the” as used herein are intended to include the plural forms as well unless expressly stated otherwise. It will be further understood that the term “including” used in the specification of the present disclosure refers to the presence of the features, integers, steps, operations, elements and/or components, but does not exclude the presence or addition of one or more other features, integers, steps, operations, elements, components, and/or groups thereof. It should be understood that when an element is referred to as being “connected” or “coupled” to another element, it can be directly connected or coupled to other elements or an intermediate element may also be present. Furthermore, “connection” or “coupling” as used herein may include wireless connection or wireless coupling. The term “and/or” as used herein includes all or any unit and all combinations of one or more associated listed items.

It will be understood by those skilled in the art that unless otherwise defined, all terms (including technical and scientific terms) used herein have the same meaning as commonly understood by those of ordinary skill in the art to which the present disclosure belongs. It will be further understood that terms such as those defined in general dictionaries should be interpreted as having meanings that are consistent with those in the context of the prior art, and will not be interpreted in an idealized or overly formal sense unless specifically defined as herein.

In view of the above defects in the prior art, the present disclosure provides a method for predicting the battery performance based on a combination of material parameters of a battery pulping process, including obtaining a target prediction model, wherein the target prediction model includes a plurality of sub-models, and the plurality of the sub-models correspond to different combinations of model parameters, respectively; and obtaining a combination of material parameters to be predicted corresponding to the battery pulping process, and inputting the combination of the material parameters to be predicted into the target prediction model to obtain a target battery performance level corresponding to the combination of the material parameters to be predicted, wherein the target battery performance level is determined according to battery performance levels output by the sub-models based on the combination of the material parameters to be predicted, respectively. The method of the present disclosure adopts a mathematical model method instead of manual testing, can quickly predict battery performance levels corresponding to different combinations of material parameters, and solves the problem that in the prior art, it is necessary to conduct separate tests for different input ratios of various materials to determine the impact of different material ratios on the battery performance, which requires a lot of manpower and time costs.

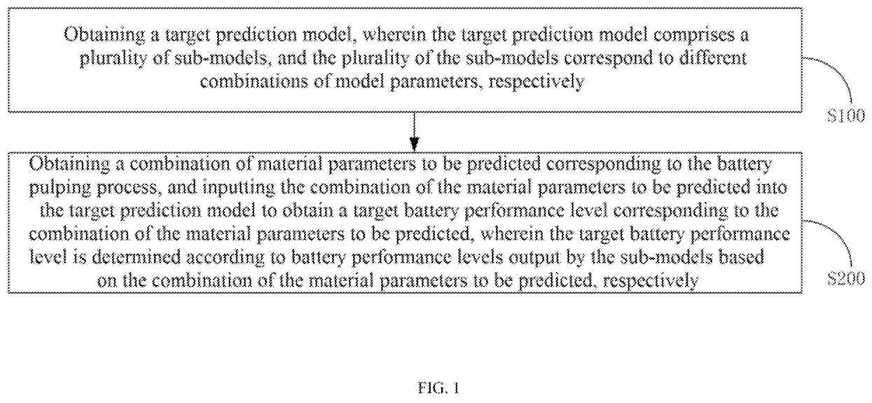

As shown in FIG. 1, the method includes the following steps:

Step S100, a target prediction model is obtained, wherein the target prediction model includes a plurality of sub-models, and the plurality of the sub-models correspond to different combinations of model parameters, respectively.

Specifically, in order to quickly determine the battery performance respectively corresponding to different combinations of material parameters in the battery pulping process, in this embodiment, a target prediction model is pre-built, wherein the target prediction model includes a plurality of sub-models each having different combinations of model parameters to perform a prediction task of the target prediction model together, thereby avoiding a problem of low reliability of output data of a single model.

In an implementation, the target prediction model is trained in advance, and a training process includes:
Step S10: a plurality of combinations of material parameters corresponding to the battery pulping process and actual battery performance levels respectively corresponding to the plurality of the combinations of the material parameters are obtained;
Step S11, one of the combinations of the material parameters is input into the target prediction model to obtain a first predicted battery performance level output by the target prediction model and a second predicted battery performance level output by each of the sub-models, wherein the first predicted battery performance level is determined based on the second predicted battery performance levels;
Step S12, a loss value corresponding to each of the sub-models is determined based on the first predicted battery performance level, the second predicted battery performance levels, and the actual battery performance level corresponding to the combination of the material parameters;
Step S13, whether the loss value corresponding to each of the sub-models converges to a target value is determined; and
Step S14, if not, a combination of initial model parameters of the sub-model is corrected according to the loss value of the sub-model not converging to the target value, and the step of inputting one of the combinations of the material parameters into the target prediction model is continued to be performed until the loss value corresponding to each of the sub-models converges to the target value to obtain the trained target prediction model.

Specifically, in order to obtain accurate prediction results, in this embodiment, the target prediction model is iteratively trained in advance with a large number of combinations of material parameters with known actual battery performance, wherein each combination of material parameters includes, but is not limited to, a specific area of lithium cobaltate particles, a specific area of graphite particles, a mass ratio of lithium cobaltate to graphite, a mass ratio of graphite to PVDF, the solid content of a slurry, the stirring time, and a stirring rate. Since the process of each round of training is similar, in this embodiment, a model training process is illustrated with one round of training as an example. For each round of training, a combination of material parameters trained in this round is input into the target prediction model, each sub-model predicts a battery performance level based on the combination of the material parameters to obtain a second battery performance level corresponding to each sub-model, and a battery performance level is comprehensively determined based on the second battery performance levels to obtain a first battery performance level output by the target prediction model. By comparing the first battery performance level and the second battery performance levels with the actual battery performance level corresponding to this round of training, a difference between an output of each sub-model and a true value can be determined to obtain a loss value of each sub-model. For each sub-model, if the loss value of the sub-model is not greater than the target value, indicating that the accuracy of the sub-model does not meet the training requirements, then the model parameters of the sub-model are corrected based on the loss value of the sub-model. When the loss values of all sub-models converge to the target value, indicating that the accuracy of the target prediction model has met the training requirements, the training is stopped.

In an implementation, the step S12 specifically includes the following steps:
Step S121, a first loss value corresponding to each of the second predicted battery performance levels is determined based on a deviation between the first predicted battery performance level and the actual battery performance level corresponding to the combination of the material parameters;
Step S122, a second loss value corresponding to each of the sub-models is determined according to a deviation between the second predicted battery performance level corresponding to each of the sub-models and the first predicted battery performance level; and
Step S123, the loss value corresponding to each of the sub-models is determined based on the first loss value and the second loss value corresponding to each of the sub-models.

Specifically, there are mainly two training objectives in this embodiment, firstly a difference between an output of the target prediction model and a true value is to be converged. Secondly, since the output of the target prediction model is comprehensively determined based on outputs of the sub-models, it is also necessary to converge a difference between the outputs of the sub-models. Thus, for each sub-model, the loss value of the sub-model is comprehensively determined by calculating a difference between the first predicted battery performance level output by the target prediction model and the actual battery performance level, and a difference between the second predicted battery performance level output by the sub-model and the first predicted battery performance level.

In an implementation, the method further includes determining a combination of target hyper-parameters corresponding to each of the sub-models before training the target prediction model, wherein a method for determining the combination of the target hyper-parameters corresponding to each of the sub-models includes:
Step S20, a plurality of combinations of hyper-parameters are obtained, wherein each of the combinations of the hyper-parameters includes a plurality of hyper-parameters of different categories;
Step S21, a plurality of hyper-parameter combination profiles are determined according to the combinations of the hyper-parameters, wherein the plurality of the hyper-parameter combination profiles are in one-to-one correspondence to the plurality of the hyper-parameters, values of target hyper-parameters corresponding to each of the combinations of the hyper-parameters in each of the hyper-parameter combination profiles are the same, and each of the combinations of the hyper-parameters is regularly distributed based on the magnitude of the values of the target hyper-parameters, and the target hyper-parameters are the hyper-parameters corresponding to the combination of the hyper-parameters;
Step S22, a combination of candidate hyper-parameters corresponding to each of the hyper-parameter combination profiles is determined, wherein each of the combinations of the hyper-parameters adjacent to the combination of the candidate hyper-parameters in each of the hyper-parameter combination profiles has a model performance level that is less than a model performance level of the combination of the candidate hyper-parameters; and
Step S23, the combination of the target hyper-parameters corresponding to each of the sub-models is determined according to the combinations of the candidate hyper-parameters.

Prior to training the model, an optimal combination of hyper-parameters, i.e., the combination of the target hyper-parameters for each sub-model, needs to be first determined. In particular, a large number of combinations of hyper-parameters are first combined based on possible values for various hyper-parameters in the sub-model. These combinations of the hyper-parameters are then categorized to obtain a plurality of sets of the combinations of the hyper-parameters, wherein values of the hyper-parameters of a specified category for each combination of the hyper-parameters in each set are the same, i.e., the values of the target hyper-parameters are the same, and different sets respectively correspond to different categories of the target hyper-parameters. It should be noted that the combinations of the hyper-parameters contained in each set may or may not overlap. For each set, a hyper-parameter combination profile is generated based on the combinations of the hyper-parameters in the set, each point in the hyper-parameter combination profile represents one combination of hyper-parameters, and a distribution law of the points conforms to a law of increasing or decreasing values of the target hyper-parameters. For each hyper-parameter combination profile, a point that satisfies a certain condition is searched in the hyper-parameter combination profile, i.e., a model performance level corresponding to each point around this point is lower than that of the point itself, and a combination of candidate hyper-parameters corresponding to the hyper-parameter combination profile is obtained through the point. Each combination of the candidate hyper-parameters represents a combination with the higher model performance level in its corresponding hyper-parameter combination profile, so that a combination of target hyper-parameters for each sub-model is determined from the combinations of the candidate hyper-parameters to improve the model effect of each sub-model. For example, combinations of hyper-parameters with model performance levels ranked first several may be selected from the combinations of the candidate hyper-parameter according to the number of sub-models to obtain the combination of the target hyper-parameters for each sub-model.

For example, assuming that hyper-parameters a, b and c are included in each sub-model, a plurality of combinations of hyper-parameters are combined according to possible values of the hyper-parameters a, b and c, and hyper-parameter combination distributions A, B and C are generated according to the combinations of the hyper-parameters. Wherein the hyper-parameter a of each combination of the hyper-parameters in the hyper-parameter combination distribution A has the same value, the hyper-parameter b of each combination of the hyper-parameters in the hyper-parameter combination distribution B has the same value, and the hyper-parameter c of each combination of the hyper-parameters in the hyper-parameter combination distribution C has the same value.

In an implementation, the step S22 specifically includes the following steps:
Step S221, anyone of the combinations of the hyper-parameters in each of the hyper-parameter combination profiles is used as a target point;
Step S222, whether model performance levels respectively corresponding to points adjacent to the target point are greater than a model performance level corresponding to the target point is determined;
Step S223, if a model performance level corresponding to any one of the points adjacent to the target point is greater than the model performance level corresponding to the target point, a next target point is determined according to a point having the highest model performance level in the points adjacent to the target point; and
Step S224, the step of determining whether the model performance levels respectively corresponding to the points adjacent to the target point are greater than the model performance level corresponding to the target point is continued to be performed until the model performance levels respectively corresponding to the points adjacent to the target point are less than the model performance level corresponding to the target point, and the combination of the hyper-parameters corresponding to the target point is used as the combination of the candidate hyper-parameters corresponding to the hyper-parameter combination profile.

In particular, for each hyper-parameter combination profile, a point corresponding to any one combination of hyper-parameters in the hyper-parameter combination profile is used as a target point, a magnitude relationship between model performance levels of points adjacent to the target point and a model performance level of the target point is then determined, if the model performance level of the target point is not the highest, a point with the highest model performance level in the adjacent points is used as a next target point, and a magnitude relationship between the model performance levels of the points adjacent to the target point and the model performance level of the target point is continued to be determined until a target point is searched such that model performance levels of points adjacent to the target point are less than a model performance level of the target point, the search is stopped, and the combination of the hyper-parameters represented by the target point finally searched is taken as the combination of the candidate hyper-parameters. It should be noted that a search mode in this embodiment does not use a traversal mode, so that the selected combination of the candidate hyper-parameters may not be an optimal combination in the hyper-parameter combination profile, but the search mode in this embodiment can significantly shorten the search time and search out a better combination.

In an implementation, the step S23 specifically includes the following steps:
Step S231, a target hyper-parameter combination profile is determined from the plurality of the hyper-parameter combination profiles based on the model performance level corresponding to each of the combinations of the candidate hyper-parameters, wherein the combination of the candidate hyper-parameters corresponding to the target hyper-parameter combination profile has the highest model performance level;
Step S232, the target hyper-parameter combination profile is traversed to obtain first several combinations of the hyper-parameters having the highest model performance level, wherein the number of the first several combinations of the hyper-parameters is determined based on the number of the sub-models; and
Step S233, the combination of the target hyper-parameters corresponding to each of the sub-models is determined in one-to-one correspondence according to the first several combinations of the hyper-parameters.

Specifically, after each combination of candidate hyper-parameters is obtained, in this embodiment, a combination of hyper-parameters with the highest model performance level is screened from the combinations of the candidate hyper-parameters, and a hyper-parameter combination profile corresponding to the combination of the hyper-parameters is re-searched in a traversal manner, and first several combinations of hyper-parameters having the highest model performance level are selected based on the number of the sub-models, and the combination of the target hyper-parameters for each sub-model is determined in one-to-one correspondence based on the selected combinations of the hyper-parameters. Since it takes too long a time to search for each hyper-parameter combination profile in a traversal manner, in this embodiment, a target hyper-parameter combination profile is selected based on a model performance level of each combination of candidate hyper-parameters, the profile is only searched in a traversal manner, and a better combination of hyper-parameters is searched for each sub-model as much as possible while shortening the overall search time.

As shown in FIG. 1, the method further includes the following steps:

Step S200, a combination of material parameters to be predicted corresponding to the battery pulping process is obtained, and the combination of the material parameters to be predicted is input into the target prediction model to obtain a target battery performance level corresponding to the combination of the material parameters to be predicted, wherein the target battery performance level is determined according to battery performance levels output by the sub-models based on the combination of the material parameters to be predicted, respectively.

Specifically, since the target prediction model is trained in advance, a correspondence between features of different input data and output data has been learned, thus, the combination of the material parameters to be predicted is input into the target prediction model, each sub-model outputs a battery performance level based on the input combination of the material parameters to be predicted, and finally the target battery performance level output by the target prediction model is determined based on the battery performance levels output by the sub-models, so as to avoid the influence of the reliability of a prediction result corresponding to a single model.

In an implementation, the target battery performance level is determined according to an average or a weighted average of the battery performance levels respectively corresponding to the sub-models.

Specifically, in order to avoid the influence of the reliability of the prediction result corresponding to the single model, in this embodiment, an output of the target prediction model is determined by using an average or a weighted average of outputs of the sub-models, thereby ensuring the reliability of the prediction result of the target prediction model.

In an implementation, since lithium cobaltate is typically used as an active material, graphite is typically used as a conductive agent, NMP (N-methylpyrrolidone) is typically used as a solvent, and PVDF (polyvinylidene fluoride) is typically used as a binder and a dispersant in the battery pulping process, the combination of the material parameters to be predicted/each combination of material parameters includes one or more of parameters such as a specific area of lithium cobaltate particles, a specific area of graphite particles, a mass ratio of lithium cobaltate to graphite, a mass ratio of graphite to PVDF, the solid content of a slurry, the stirring time and a stirring rate. For the actual battery performance level corresponding to each of the combinations of the material parameters, the active material and the conductive agent are dried and mixed, and the binder and the solvent are added according to the combination of the material parameters. The viscosity of the resulting slurry, the compression performance of a finished pole piece, and the charge-discharge cycling performance of a half-cell are then tested, and the actual battery performance level corresponding to the combination of the material parameters is comprehensively determined based on the viscosity of the slurry, the compression performance, and the charge-discharge cycling performance of the half-cell.

In an implementation, there are a plurality of the combinations of the material parameters to be predicted, a combination of material parameters with the highest battery performance level in the plurality of the combinations of the material parameters to be predicted is used as a combination of target material parameters, and operating parameters in the battery pulping process are determined according to the combination of the target material parameters.

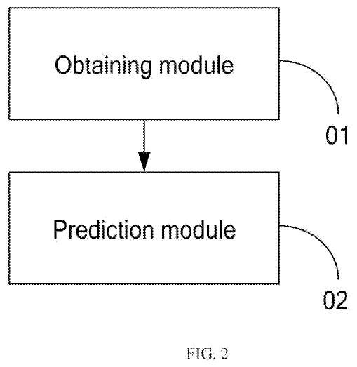

Based on the above embodiment, the present disclosure further provides an apparatus for predicting the battery performance based on a combination of material parameters of a battery pulping process, wherein as shown in FIG. 2, the apparatus includes:
an obtaining module 01, configured to obtain a target prediction model, wherein the target prediction model includes a plurality of sub-models, and the plurality of the sub-models correspond to different combinations of model parameters, respectively; and
a prediction module 02, configured to obtain a combination of material parameters to be predicted corresponding to the battery pulping process, and input the combination of the material parameters to be predicted into the target prediction model to obtain a target battery performance level corresponding to the combination of the material parameters to be predicted, wherein the target battery performance level is determined according to battery performance levels output by the sub-models based on the combination of the material parameters to be predicted, respectively.

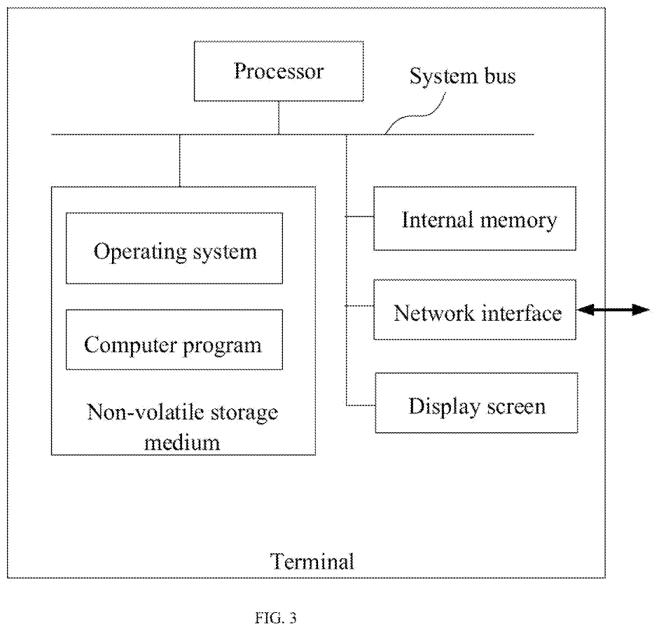

Based on the above embodiments, the present disclosure further provides a terminal, a functional block diagram of which may be as shown in FIG. 3. The terminal includes a processor, a memory, a network interface, and a display screen which are connected by a system bus. Wherein the processor of the terminal is configured to provide computing and control capabilities. The memory of the terminal includes a non-volatile storage medium, and an internal memory. The non-volatile storage medium stores an operating system and a computer program. The internal memory provides an environment for the running of the operating system and the computer program in the non-volatile storage medium. The network interface of the terminal is configured to communicate with an external terminal via a network connection. The computer program, when executed by a processor, implements the method for predicting the battery performance based on a combination of material parameters of a buttery pulping process. The display screen of the terminal may be a liquid crystal display screen or an e-ink display screen.

Those skilled in the art can understand that the functional block diagram shown in FIG. 3 is merely a block diagram of a partial structure related to the solution of the present disclosure, and does not constitute a limitation of a terminal to which the solution of the present disclosure is applied, and a specific terminal may include more or fewer components than those shown in the figure, or combine some components, or have a different arrangement of components.

In an implementation, one or more programs are stored in the memory of the terminal and configured to be executed by one or more processors, the one or more programs include instructions for performing the method for predicting the battery performance based on a combination of material parameters of a battery pulping process.

Those of ordinary skill in the art can understand that implementing all or part of the processes in the method of the above embodiment may be completed by instructing the relevant hardware through a computer program, the computer program can be stored in a non-volatile computer-readable storage medium, and the computer program, when executed, may include the processes in the embodiments of the methods described above. Any reference to a memory, storage, a database, or other media used in the embodiments provided in the present disclosure may include non-volatile and/or volatile memories. The non-volatile memory may include a read-only memory (ROM), a programmable ROM (PROM), an electrically programmable ROM (EPROM), an electrically erasable programmable ROM (EEPROM), or a flash memory. The volatile memory may include a random access memory (RAM) or an external cache memory.

By way of illustration and not limitation, the RAM is available in various forms such as a static RAM (SRAM), a dynamic RAM (DRAM), a synchronous DRAM (SDRAM), a double data rate SDRAM (DDRSDRAM), an enhanced SDRAM (ESDRAM), a synchlink DRAM (SLDRAM), a Rambus direct RAM (RDRAM), a direct Rambus dynamic RAM (DRDRAM), and a Rambus dynamic RAM (RDRAM).

In summary, the present disclosure discloses the method for predicting the battery performance based on a combination of material parameters of a battery pulping process, including obtaining the target prediction model, wherein the target prediction model includes the plurality of the sub-models, and the plurality of the sub-models correspond to different combinations of model parameters, respectively; and obtaining the combination of the material parameters to be predicted corresponding to the battery pulping process, and inputting the combination of the material parameters to be predicted into the target prediction model to obtain the target battery performance level corresponding to the combination of the material parameters to be predicted, wherein the target battery performance level is determined according to the battery performance levels output by the sub-models based on the combination of the material parameters to be predicted, respectively. The method of the present disclosure adopts a mathematical model method instead of manual testing, can quickly predict battery performance levels corresponding to different combinations of material parameters, and solves the problem that in the prior art, it is necessary to conduct separate tests for different input ratios of various materials to determine the impact of different material ratios on the battery performance, which requires a lot of manpower and time costs.

It should be understood that the application of the present disclosure is not limited to the examples described above, and for those of ordinary skill in the art, modifications and variations may be made according to the above description, and all these improvements and variations are intended to fall within the protection scope of the appended claims of the present disclosure.

What is claimed is:

1. A method for predicting the battery performance based on a combination of material parameters of a battery pulping process, comprising:
obtaining a target prediction model, wherein the target prediction model comprises a plurality of sub-models, and the plurality of the sub-models correspond to different combinations of model parameters, respectively; and
obtaining a combination of material parameters to be predicted corresponding to the battery pulping process, and inputting the combination of the material parameters to be predicted into the target prediction model to obtain a target battery performance level corresponding to the combination of the material parameters to be predicted, wherein the target battery performance level is determined according to battery performance levels output by the sub-models based on the combination of the material parameters to be predicted, respectively.

2. The method for predicting the battery performance based on a combination of material parameters of a battery pulping process according to claim 1, wherein the target prediction model is trained in advance, and a training process comprises:
obtaining a plurality of combinations of material parameters corresponding to the battery pulping process and actual battery performance levels respectively corresponding to the plurality of the combinations of the material parameters;
inputting one of the combinations of the material parameters into the target prediction model to obtain a first predicted battery performance level output by the target prediction model and a second predicted battery performance level output by each of the sub-models, wherein the first predicted battery performance level is determined based on the second predicted battery performance levels;
determining a loss value corresponding to each of the sub-models based on the first predicted battery performance level, the second predicted battery performance levels, and the actual battery performance level corresponding to the combination of the material parameters;
determining whether the loss value corresponding to each of the sub-models converges to a target value; and
if not, correcting a combination of initial model parameters of the sub-model according to the loss value of the sub-model not converging to the target value, and continuing to perform the step of inputting one of the combinations of the material parameters into the target prediction model until the loss value corresponding to each of the sub-models converges to the target value to obtain the trained target prediction model.

3. The method for predicting the battery performance based on a combination of material parameters of a battery pulping process according to claim 2, wherein determining the loss value corresponding to each of the sub-models based on the first predicted battery performance level, the second predicted battery performance levels, and the actual battery performance level corresponding to the combination of the material parameters comprises:
determining a first loss value corresponding to each of the second predicted battery performance levels based on a deviation between the first predicted battery performance level and the actual battery performance level corresponding to the combination of the material parameters;
determining a second loss value corresponding to each of the sub-models according to a deviation between the second predicted battery performance level corresponding to each of the sub-models and the first predicted battery performance level; and
determining the loss value corresponding to each of the sub-models based on the first loss value and the second loss value corresponding to each of the sub-models.

4. The method for predicting the battery performance based on a combination of material parameters of a battery pulping process according to claim 2, further comprising determining a combination of target hyper-parameters corresponding to each of the sub-models before training the target prediction model, wherein a method for determining the combination of the target hyper-parameters corresponding to each of the sub-models comprises:
obtaining a plurality of combinations of hyper-parameters, wherein each of the combinations of the hyper-parameters comprises a plurality of hyper-parameters of different categories;
determining a plurality of hyper-parameter combination profiles according to the combinations of the hyper-parameters, wherein the plurality of the hyper-parameter combination profiles are in one-to-one correspondence to the plurality of the hyper-parameters, values of target hyper-parameters corresponding to each of the combinations of the hyper-parameters in each of the hyper-parameter combination profiles are the same, and each of the combinations of the hyper-parameters is regularly distributed based on the magnitude of the values of the target hyper-parameters, and the target hyper-parameters are the hyper-parameters corresponding to the combination of the hyper-parameters;
determining a combination of candidate hyper-parameters corresponding to each of the hyper-parameter combination profiles, wherein each of the combinations of the hyper-parameters adjacent to the combination of the candidate hyper-parameters in each of the hyper-parameter combination profiles has a model performance level that is less than a model performance level of the combination of the candidate hyper-parameters; and
determining the combination of the target hyper-parameters corresponding to each of the sub-models according to the combinations of the candidate hyper-parameters.

5. The method for predicting the battery performance based on a combination of material parameters of a battery pulping process according to claim 4, wherein determining the combination of the candidate hyper-parameters corresponding to each of the hyper-parameter combination profiles comprises:
determining a target point based on one of the combinations of the hyper-parameters in each of the hyper-parameter combination profiles;
determining whether model performance levels respectively corresponding to points adjacent to the target point are greater than a model performance level corresponding to the target point;
if a model performance level corresponding to any one of the points adjacent to the target point is greater than the model performance level corresponding to the target point, determining a next target point according to a point having the highest model performance level in the points adjacent to the target point; and
continuing to perform the step of determining whether the model performance levels respectively corresponding to the points adjacent to the target point are greater than the model performance level corresponding to the target point until the model performance levels respectively corresponding to the points adjacent to the target point are less than the model performance level corresponding to the target point, and using the combination of the hyper-parameters corresponding to the target point as the combination of the candidate hyper-parameters corresponding to the hyper-parameter combination profile.

6. The method for predicting the battery performance based on a combination of material parameters of a battery pulping process according to claim 4, wherein determining the combination of the target hyper-parameters corresponding to each of the sub-models according to the combinations of the candidate hyper-parameters comprises:
determining a target hyper-parameter combination profile from the plurality of the hyper-parameter combination profiles based on the model performance level corresponding to each of the combinations of the candidate hyper-parameters, wherein the combination of the candidate hyper-parameters corresponding to the target hyper-parameter combination profile has the highest model performance level;
traversing the target hyper-parameter combination profile to obtain first several combinations of the hyper-parameters having the highest model performance level, wherein the number of the first several combinations of the hyper-parameters is determined based on the number of the sub-models; and
determining, in one-to-one correspondence, the combination of the target hyper-parameters corresponding to each of the sub-models according to the first several combinations of the hyper-parameters.

7. The method for predicting the battery performance based on a combination of material parameters of a battery pulping process according to claim 1, wherein the target battery performance level is determined according to an average or a weighted average of the battery performance levels respectively corresponding to the sub-models.

8. An apparatus for predicting the battery performance based on a combination of material parameters of a battery pulping process, comprising:
an obtaining module, configured to obtain a target prediction model, wherein the target prediction model comprises a plurality of sub-models, and the plurality of the sub-models correspond to different combinations of model parameters, respectively; and
a prediction module, configured to obtain a combination of material parameters to be predicted corresponding to the battery pulping process, and input the combination of the material parameters to be predicted into the target prediction model to obtain a target battery performance level corresponding to the combination of the material parameters to be predicted, wherein the target battery performance level is determined according to battery performance levels output by the sub-models based on the combination of the material parameters to be predicted, respectively.

9. A terminal, comprising a memory and one or more processors; wherein the memory stores one or more programs; and the programs are adapted to be loaded and executed by the processors to:
obtain a target prediction model, wherein the target prediction model comprises a plurality of sub-models, and the plurality of the sub-models correspond to different combinations of model parameters, respectively; and
obtain a combination of material parameters to be predicted corresponding to a battery pulping process, and input the combination of the material parameters to be predicted into the target prediction model to obtain a target battery performance level corresponding to the combination of the material parameters to be predicted, wherein the target battery performance level is determined according to battery performance levels output by the sub-models based on the combination of the material parameters to be predicted, respectively.
